(19) United States
(12) Patent Application Publication
(10) Pub. No.: US 2024/0288020 A1
(43) Pub. Date: Aug. 29, 2024

(54) FASTENER, METHOD OF MANUFACTURING ULTRASOUND TREATMENT TOOL, AND ULTRASOUND TREATMENT TOOL

(71) Applicant: OLYMPUS MEDICAL SYSTEMS CORP., Tokyo (JP)

(72) Inventors: Yasuhiro MAEDA, Tokyo (JP); Yukihiko SAWADA, Yoshikawa-shi (CN); Hideto YOSHIMINE, Tokorozawa-shi (CN); Minoru KATSUMATA, Tokyo (JP); Hiroshi ASHIBA, Tokyo (JP); Shunsuke MATSUI, Tokyo (JP)

(21) Appl. No.: 18/658,395

(22) Filed: May 8, 2024

Related U.S. Application Data
(63) Continuation of application No. PCT/JP2021/041416, filed on Nov. 10, 2021.

(51) Int. Cl. F16B 31/02; A61B 17/00; A61B 17/32

(52) U.S. Cl. CPC F16B 31/02; A61B 17/320092; A61B 2017/00526; A61B 2017/320082; A61B 2017/320095; F16B 2031/022

(57) ABSTRACT

A fastener includes: a main body including a first fastening portion configured to be screwed together with a ultrasound transducer configured to generate ultrasound vibration, and a second fastening portion configured to be screwed together with a vibration transmission portion configured to transmit the ultrasound vibration, the main body being configure to connect the ultrasound transducer and the vibration transmission portion; and a rupture portion configured to be ruptured as a result of a specific torque being applied when the first fastening portion is screwed together with the ultrasound transducer or when the second fastening portion is screwed together with the vibration transmission portion.

### CROSS-REFERENCE TO RELATED APPLICATION

This application is a continuation of International Application No. PCT/JP2021/041416, filed on Nov. 10, 2021, the entire contents of which are incorporated herein by reference.

### BACKGROUND

### 1. Technical Field

The present disclosure relates to a fastener, a method of manufacturing an ultrasound treatment tool, and the ultrasound treatment tool.

### 2. Related Art

In the related art, there is a known ultrasound treatment tool that performs treatment on a region targeted for treatment of biological tissue (hereinafter, referred to as a target region) by using ultrasound vibration (for example, see Japanese Patent No. 6067208).

The ultrasound treatment tool described in Japanese Patent No. 6067208 includes an ultrasound transducer that generates ultrasound vibration, a vibration transmission portion that transmits the ultrasound vibration, and a fastener that is screwed together with each of the ultrasound transducer and the vibration transmission portion, and that connects the ultrasound transducer and the vibration transmission portion.

### SUMMARY

In some embodiments, a fastener includes: a main body including a first fastening portion configured to be screwed together with a ultrasound transducer configured to generate ultrasound vibration, and a second fastening portion configured to be screwed together with a vibration transmission portion configured to transmit the ultrasound vibration, the main body being configure to connect the ultrasound transducer and the vibration transmission portion; and a rupture portion configured to be ruptured as a result of a specific torque being applied when the first fastening portion is screwed together with the ultrasound transducer or when the second fastening portion is screwed together with the vibration transmission portion. In a state in which the first fastening portion is screwed together with the ultrasound transducer, an end surface of the first fastening portion is not in contact with a bottom surface of a fastening portion provided at the ultrasound transducer, and in a state in which the second fastening portion is screwed together with the vibration transmission portion, an end surface of the second fastening portion is in contact with a bottom surface of a fastening portion provided at the vibration transmission portion.

In some embodiments, provided is a method of manufacturing an ultrasound treatment tool that includes an ultrasound transducer configured to generate ultrasound vibration, a vibration transmission portion configured to transmit the ultrasound vibration, and a fastener that connects the ultrasound transducer and the vibration transmission portion. The method includes: screwing the fastener together with the vibration transmission portion; rupturing the fastener by applying a specific torque to the fastener when the fastener is screwed together with the vibration transmission portion; and screwing the fastener together with the ultrasound transducer.

In some embodiments, an ultrasound treatment tool includes: an ultrasound transducer configured to generate ultrasound vibration; a vibration transmission portion configured to transmit the ultrasound vibration; and a fastener configured to connect the ultrasound transducer and the vibration transmission portion. The fastener includes a main body that includes a first fastening portion configured to be screwed together with the ultrasound transducer, and a second fastening portion configured to be screwed together with the vibration transmission portion. An end surface of the first fastening portion is not in contact with a bottom surface of a fastening portion provided at the ultrasound transducer, and an end surface of the second fastening portion is in contact with a bottom surface of a fastening portion provided at the vibration transmission portion.

The above and other features, advantages and technical and industrial significance of this disclosure will be better understood by reading the following detailed description of presently preferred embodiments of the disclosure, when considered in connection with the accompanying drawings.

### BRIEF DESCRIPTION OF THE DRAWINGS

FIG. 1 is a diagram illustrating a treatment system according to a first embodiment;

FIG. 2 is a diagram illustrating a configuration of a vibration transmission portion;

FIG. 3 is a diagram illustrating a configuration of a fastener;

FIG. 4 is a diagram illustrating a configuration of the fastener;

FIG. 5 is a diagram illustrating a modification 1-1 according to the first embodiment;

FIG. 6 is a diagram illustrating a modification 1-2 according to the first embodiment;

FIG. 7 is a diagram illustrating a modification 1-3 according to the first embodiment;

FIG. 8 is a diagram illustrating the modification 1-3 according to the first embodiment;

FIG. 9 is a diagram illustrating a modification 1-4 according to the first embodiment;

FIG. 10 is a diagram illustrating the modification 1-4 according to the first embodiment;

FIG. 11 is a diagram illustrating a fastener according to a second embodiment;

FIG. 12 is a diagram illustrating a modification 2-1 according to the second embodiment;

FIG. 13 is a diagram illustrating a modification 2-2 according to the second embodiment;

FIG. 14 is a diagram illustrating a modification 2-3 according to the second embodiment; and

FIG. 15 is a diagram illustrating a modification 2-4 according to the second embodiment.

### DETAILED DESCRIPTION

Modes (hereinafter, embodiments) for carrying out the disclosure will be described below with reference to the drawings. Furthermore, the disclosure is not limited to the embodiments described below. In addition, in description of the drawings, components that are identical to those in drawings are assigned the same reference numerals.

### First Embodiment

### Schematic Configuration of Treatment System

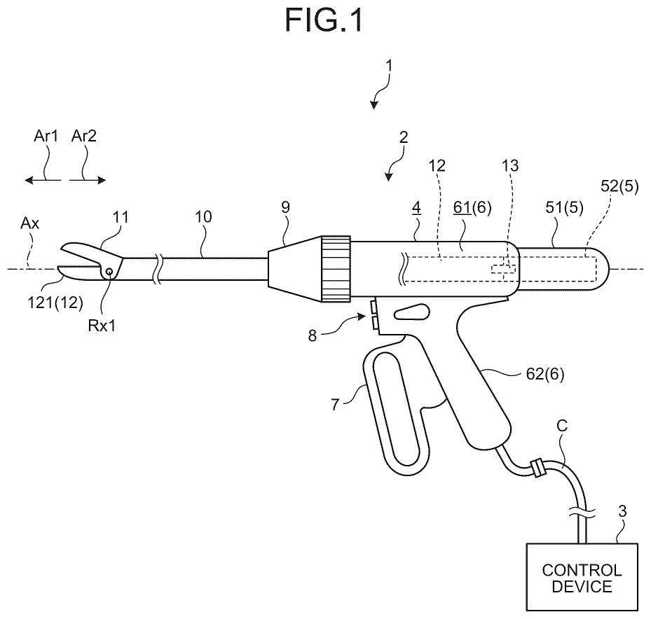

FIG. 1 is a diagram illustrating a treatment system 1 according to an embodiment.

The treatment system 1 performs treatment on a region targeted for treatment of biological tissue (hereinafter, referred to as a target region) by applying treatment energy to the target region. Furthermore, the treatment energy according to the present embodiment is ultrasound energy and high frequency energy. Moreover, the treatment that is able to be performed by the treatment system 1 according to the present embodiment is treatment including coagulation (sealing) of the target region, incision of the target region, or the like. Moreover, it may be possible to use a configuration in which both coagulation and incision are performed at the same time. The treatment system 1 includes, as illustrated in FIG. 1, an ultrasound treatment tool 2 and a control device 3.

### Configuration of Ultrasound Treatment Tool

In the following description of the configuration of the ultrasound treatment tool 2, one of the sides along a central axis Ax of a shaft 10 is referred to as a distal end side Ar1, whereas the other of the sides is referred to as a proximal end side Ar2.

The ultrasound treatment tool 2 is an ultrasound treatment tool that performs treatment on a target region by applying ultrasound energy and high frequency energy to the target region. The ultrasound treatment tool 2 includes, as illustrated in FIG. 1, a handpiece 4 and an ultrasonic transducer unit 5.

The handpiece 4 includes, as illustrated in FIG. 1, a housing 6, a movable handle 7, switches 8, a rotatable knob 9, the shaft 10, a jaw 11, a vibration transmission portion 12, and a fastener 13.

The housing 6 supports the entire of the ultrasound treatment tool 2. The housing 6 includes, as illustrated in FIG. 1, a case body 61 that is formed of a substantially cylindrical shape and that is coaxial with the central axis Ax, and a fixed handle 62 that extends from the case body 61 in the downward direction in the drawing illustrated in FIG. 1 and that is to be gripped by an operator, such as a technician.

The movable handle 7 receives each of a closing operation and an opening operation that are performed by the operator, such as the technician. Then, the movable handle 7 moves in a direction closer to the fixed handle 62 in response to the closing operation. In contrast, the movable handle 7 moves in a direction away from the fixed handle 62 in response to the opening operation.

As illustrated in FIG. 1, each of the switches 8 is externally exposed from the side surface of the fixed handle 62 located on the distal end side Ar1. Then, each of the switches 8 receives a treatment operation performed by the operator, such as the technician. The treatment operation is an operation for applying treatment energy to the target region.

The rotatable knob 9 is formed to have a substantially cylindrical shape that is coaxial with the central axis Ax, and is provided at, as illustrated in FIG. 1, the case body 61 on the distal end side Ar1. Then, the rotatable knob 9 receives a rotation operation performed by the operator, such as the technician. As a result of the rotation operation, the rotatable knob 9 is rotated about the central axis Ax relative to the case body 61. Furthermore, as a result of the rotation of the rotatable knob 9, the shaft 10, the jaw 11, and the vibration transmission portion 12 are also rotated about the central axis Ax.

The shaft 10 is a pipe that is formed in a cylindrical shape and that is constituted by a material, such as metal.

At the end portion of the shaft 10 on the distal end side Ar1, the jaw 11 is axially supported so as to be rotatable about the rotation axis Rx1 that extends in the direction orthogonal to the plane of the drawing in FIG. 1.

Here, although a specific illustration has been omitted, an opening/closing mechanism that allows the jaw 11 to be rotated about the rotation axis Rx1 in response to the opening operation and the closing operation that are performed on the movable handle 7 by the operator, such as the technician, is provided in the internal portion of the housing 6 and the shaft 10.

Then, the jaw 11 is opened and closed with respect to an end portion 121 (hereinafter, referred to as a treatment portion 121 (FIG. 1)) of the vibration transmission portion 12 located on the distal end side Ar1 by the opening/closing mechanism, and grips the target region between the treatment portion 121 and the jaw 11.

The vibration transmission portion 12 has a long shape that is constituted by an electrically conductive material, and that linearly extends along the central axis Ax. Furthermore, the vibration transmission portion 12 is inserted into, as illustrated in FIG. 1, the interior of the shaft 10 in a state in which the treatment portion 121 externally protrudes. At this time, the end portion of the vibration transmission portion 12 located on the proximal end side Ar2 is mechanically connected to an ultrasound transducer 52 that constitutes the ultrasonic transducer unit 5 by the fastener 13.

Then, the vibration transmission portion 12 transmits the ultrasound vibration that has been generated by the ultrasonic transducer unit 5 from the end portion of the vibration transmission portion 12 located on the proximal end side Ar2 to the treatment portion 121. In the first embodiment, the ultrasound vibration is longitudinal vibration that vibrates in a direction along the central axis Ax.

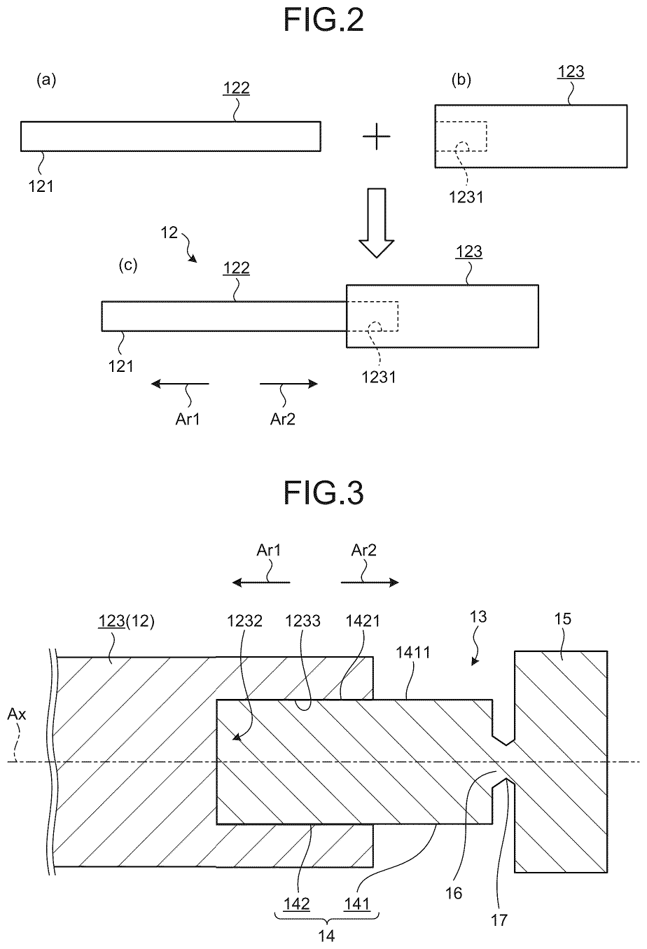

FIG. 2 is a diagram illustrating a configuration of the vibration transmission portion 12.

The vibration transmission portion 12 described above is constituted by, as illustrated in FIG. 2, two members corresponding to a small diameter portion 122 and a large diameter portion 123.

The small diameter portion 122 is a portion in which the end portion of the distal end side Ar1 corresponds to the treatment portion 121, is made of, for example, a titanium alloy, and has a columnar shape that linearly extends along the central axis Ax.

The large diameter portion 123 is made of, for example, an aluminum alloy, and has a columnar shape that linearly extends along the central axis Ax. Here, a recess portion 1231 that is recessed toward the proximal end side Ar2 is provided on the end surface of the large diameter portion 123 located on the distal end side Ar1 (FIG. 2). Then, the end portion of the small diameter portion 122 located on the proximal end side Ar2 is fixed while being inserted into the recess portion 1231. An example of the fixing method includes a fixing method, such as shrink fitting or press fit, (for example, see International Publication Pamphlet No. WO 2019/116510), or a fixing method performed by using a screw or an adhesive agent.

The fastener 13 is a member that connects the vibration transmission portion 12 and the ultrasound transducer 52.

Furthermore, a detailed configuration of the fastener 13 will be described later in a “configuration of fastener”.

The ultrasonic transducer unit 5 includes, as illustrated in FIG. 1, a transducer (TD) case 51 and the ultrasound transducer 52.

The TD case 51 supports the ultrasound transducer 52, and is connected to the case body 61 so as to be capable of being attached to and removed from the case body 61.

The ultrasound transducer 52 generates ultrasound vibration under the control of the control device 3. In the first embodiment, the ultrasound transducer 52 is constituted by a bolt-clamped Langevin-type transducer (BLT).

### Configuration of Control Device

The control device 3 performs overall control of the ultrasound treatment tool 2 by way of an electric cable C (FIG. 1).

Specifically, the control device 3 detects the treatment operation performed on the switch 8 by the operator, such as the technician, by way of the electric cable C. Then, if the control device 3 has detected the treatment operation, the control device 3 applies, by way of the electric cable C, treatment energy to the target region that is gripped between the jaw 11 and the treatment portion 121. In other words, the control device 3 performs treatment on the target region.

For example, when the control device 3 applies ultrasound energy to the target region, the control device 3 supplies driving power to the ultrasound transducer 52 by way of the electric cable C. As a result of this, the ultrasound transducer 52 generates longitudinal vibration (ultrasound vibration) that vibrates in the direction along the central axis Ax. Furthermore, the treatment portion 121 vibrates at a desired amplitude by the longitudinal vibration. Then, the ultrasound vibration is applied from the treatment portion 121 to the target region that is gripped between the jaw 11 and the treatment portion 121. In other words, the ultrasound energy is applied from the treatment portion 121 to the target region.

Furthermore, for example, when high frequency energy is applied to the target region, the control device 3 supplies high frequency power between the jaw 11 and the vibration transmission portion 12 by way of the electric cable C. As a result of this, a high frequency current flows in the target region that is gripped between the jaw 11 and the treatment portion 121. In other words, the high frequency energy is applied to the target region. In this case, at least a part of the jaw 11 is a conductive body. Furthermore, a current line may be constituted by using the shaft 10 as a conductive material.

### Configuration of Fastener

In the following, a configuration of the fastener 13 will be described.

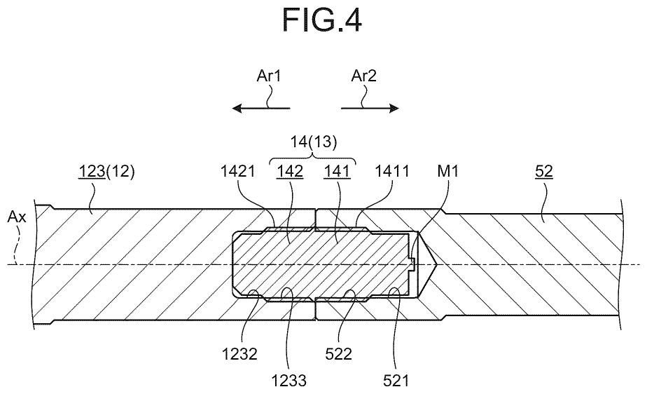

FIG. 3 and FIG. 4 are diagrams each explaining a configuration of the fastener 13. Specifically, FIG. 3 is a cross-sectional view of the fastener 13 that is in a state before a completion of manufacturing (assembling) the ultrasound treatment tool 2, and that is in a state in which the fastener 13 has been screwed together with the vibration transmission portion 12. FIG. 4 is a cross-sectional view of the fastener 13 that is in a state after the completion of manufacturing (assembling) the ultrasound treatment tool 2, and that is in a state in which the vibration transmission portion 12 and the ultrasound transducer 52 have been connected by the fastener 13.

Here, as illustrated in FIG. 4, a recess portion 521 that is recessed toward the proximal end side Ar2 is provided on the end surface of the ultrasound transducer 52 located on the distal end side Ar1. The recess portion 521 has a circular shape in cross section that is obtained by cutting the recess portion 521 by a plane orthogonal to the central axis Ax and that is coaxial with the central axis Ax, and has a substantially the same cross-sectional shape over the entire length along the central axis Ax. Furthermore, a screw groove 522 (FIG. 4) is provided on the inner circumferential surface of the recess portion 521. In other words, the recess portion 521 corresponds to a female screw portion. In a description below, for convenience of description, the recess portion 521 is referred to as a first female screw portion 521.

Furthermore, as illustrated in FIG. 3 or FIG. 4, a recess portion 1232 that is recessed toward the distal end side Ar1 is provided on the end surface of the large diameter portion 123 located on the proximal end side Ar2. The recess portion 1232 has a circular shape in cross section that is obtained by cutting the recess portion 1232 by a plane orthogonal to the central axis Ax and that is coaxial with the central axis Ax, and has a substantially the same cross-sectional shape over the entire length along the central axis Ax. Furthermore, an inner diameter size of the recess portion 1232 is substantially the same as the inner diameter size of the first female screw portion 521. Furthermore, a screw groove 1233 is provided on the inner circumferential surface of the recess portion 1232 (FIG. 3 and FIG. 4). In other words, the recess portion 1232 corresponds to a female screw portion. In a description below, for convenience of description, the recess portion 1232 is referred to as a second female screw portion 1232.

The fastener 13 includes, as illustrated in FIG. 3 or FIG. 4, a main body 14, a tool gripping portion 15 (FIG. 3), and a connecting portion 16 (FIG. 3). The main body 14, the tool gripping portion 15, and the connecting portion 16 are integrally constituted by a material having predetermined rigidity, such as a titanium alloy.

The main body 14 is coaxial with the central axis Ax, and has a columnar shape that extends along the central axis Ax.

In the main body 14, a portion of the proximal end side Ar2 functions as a first fastening portion 141 (FIG. 3 and FIG. 4) that is screwed together with the first female screw portion 521 in the ultrasound transducer 52. In other words, a screw groove 1411 is provided on the outer circumferential surface of the first fastening portion 141 (FIG. 3 and FIG. 4). The first fastening portion 141 corresponds to a male screw portion.

Furthermore, in the main body 14, a portion of the distal end side Ar1 functions as a second fastening portion 142 that is screwed together with the second female screw portion 1232 in the vibration transmission portion 12. In other words, a screw groove 1421 is provided on the outer circumferential surface of the second fastening portion 142 (FIG. 3 and FIG. 4). The second fastening portion 142 corresponds to a male screw portion.

As described above, the main body 14 has the same shape as what is called as a locking screw.

The tool gripping portion 15 is arranged at a position opposite to the end surface of the main body 14 located on the proximal end side Ar2, and has a columnar shape that is coaxial with the main body 14. The tool gripping portion 15 functions as what is called as a screw head, and is a portion that is gripped by a tool when the second fastening portion 142 is screwed together with the second female screw portion 1232.

The connecting portion 16 is a portion that connects the main body 14 and the tool gripping portion 15, is arranged between the main body 14 and the tool gripping portion 15, and has a columnar shape that is coaxial with the main body 14.

The cross-sectional areas of the cross sections of the main body 14, the tool gripping portion 15, and the connecting portion 16 described above obtained by cutting the main body 14, the tool gripping portion 15, and the connecting portion 16 by a plane orthogonal to the central axis Ax are smaller in the order of the tool gripping portion 15, the main body 14, and the connecting portion 16.

Then, in the first embodiment, a rupture portion 17 is provided at the connecting portion 16 (FIG. 3).

The rupture portion 17 is a portion that is ruptured as a result of a specific torque being applied to the rupture portion 17 when the second fastening portion 142 is screwed together with the second female screw portion 1232. In the first embodiment, the rupture portion 17 is constituted by a groove that is formed by cutting out a part of the connecting portion 16. More specifically, the rupture portion 17 corresponding to the groove is provided around the entire circumference of the outer circumferential surface of the connecting portion 16 in the circumferential direction with the central axis Ax as a center.

### Method of Manufacturing Ultrasound Treatment Tool

In the following, a method of manufacturing (assembly method) the ultrasound treatment tool 2 described above will be described with reference to FIG. 3 and FIG. 4.

Furthermore, in a description below, a step of connecting the vibration transmission portion 12 and the ultrasound transducer 52 by using the fastener 13 will mainly be described.

First, as illustrated in FIG. 3, a worker screws the second fastening portion 142 together with the second female screw portion 1232. Then, the worker rotates the fastener 13 about the central axis Ax with respect to the vibration transmission portion 12 while gripping the tool gripping portion 15, and tighten the fastener 13 with respect to the vibration transmission portion 12. At this time, if a specific torque is applied to the rupture portion 17, the rupture portion 17 is ruptured. In addition, a rupture mark M1 is provided on an outer surface of the main body 14 (FIG. 4). Furthermore, in this state, as illustrated in FIG. 3 or FIG. 4, the end surface of the second fastening portion 142 located on the distal end side Ar1 enters a state in which the end surface is in contact with the bottom surface of the second female screw portion 1232.

Furthermore, the tool that is to be used the above described process is not limited to a tool for exclusive use, such as a torque wrench, and another tool may be used.

Then, the worker inserts the vibration transmission portion 12, to which the fastener 13 is fastened, into the interior of the housing 6, and then assembles the handpiece 4.

Then, the worker attaches the ultrasonic transducer unit 5 to the case body 61.

Then, the worker connects, by using the torque wrench, the vibration transmission portion 12 and the ultrasound transducer 52 (for example, see Japanese Patent No. 4675437).

Specifically, the worker allows the torque wrench to be engaged with the rotatable knob 9. Furthermore, the worker rotates the rotatable knob 9 about the central axis Ax by using the torque wrench. As a result of this, the vibration transmission portion 12 is rotated about the central axis Ax together with the rotatable knob 9. Then, the first fastening portion 141 in the fastener 13 that has been fastened to the vibration transmission portion 12 is screwed together with the first female screw portion 521, and is fastened to the first female screw portion 521 at a specified torque. In this state, the end surface of the vibration transmission portion 12 located on the proximal end side Ar2 and the end surface of the ultrasound transducer 52 located on the distal end side Ar1 abut against each other, and, as illustrated in FIG. 4, the end surface of the first fastening portion 141 located on the proximal end side Ar2 is not in contact with the bottom surface of the first female screw portion 521.

According to the first embodiment described above, the following advantages are provided.

The fastener 13 according to the first embodiment includes the rupture portion 17 that is ruptured as a specific torque being applied to the rupture portion 17 when the second fastening portion 142 is screwed together with the vibration transmission portion 12. As a result of this, the fastener 13 is fastened to the vibration transmission portion 12 by a desired torque.

Therefore, with the fastener 13 according to the first embodiment, the performance of treatment performed on the target region is not changed from the desired performance, and thus, it is possible to suppress an individual variation in the performance that possibly occurs for each manufactured product of the ultrasound treatment tool 2, and it is thus possible to stabilize the performance.

Incidentally, in the case where the same configuration as that of the first fastening portion 141 included in the fastener 13 is provided with respect to the large diameter portion 123, it is assumed a case in which the ultrasound transducer 52 is assembled to the vibration transmission portion 12, or, it is assumed a case in which the ultrasound transducer 52 is removed from the vibration transmission portion 12. In this case, the large diameter portion 123 is made of an aluminum alloy that has relatively low strength, so that a part of the same configuration as that of the first fastening portion 141 provided in the large diameter portion 123 is raptured and may sometimes remain in the ultrasound transducer 52. Then, in such a case, there is a need to perform a work to remove the remaining portion from the ultrasound transducer 52.

In contrast, the fastener 13 according to the first embodiment is constituted by being separated from the large diameter portion 123, and is constituted by a member that has relatively higher strength than that of the large diameter portion 123 and that is made of, for example, a titanium alloy or the like, so that, in a case in which the ultrasound transducer 52 is assembled to the vibration transmission portion 12, or in a case in which the ultrasound transducer 52 is removed from the vibration transmission portion 12, it is possible to prevent the first fastening portion 141 from being ruptured and remaining in the ultrasound transducer 52.

Furthermore, in the case where a configuration in which, as with the ultrasound treatment tool described in Japanese Patent No. 6067208, the vibration transmission portion 12 and the ultrasound transducer 52 are connected by the fastener 13 is used, it is assumed a case in which the ultrasound transducer 52 is removed from the vibration transmission portion 12. In this case, if the fastener 13 is not fastened to the vibration transmission portion 12 by a desired torque, there may be a case in which a connection between the ultrasound transducer 52 and the fastener 13 is separated first before a connection between the vibration transmission portion 12 and the fastener 13 is separated, and the fastener 13 consequently remains in the ultrasound transducer 52. Then, there is a need to perform a work to remove the fastener 13 from the ultrasound transducer 52.

In contrast, the fastener 13 according to the first embodiment includes the rupture portion 17 that is ruptured as a result of a specific torque being applied to the rupture portion 17 when the second fastening portion 142 is screwed together with the vibration transmission portion 12.

Accordingly, the fastener 13 is fastened to the vibration transmission portion 12 by the desired torque. Here, setting is performed such that the desired torque that is the fastening torque between the vibration transmission portion 12 and the fastener 13 is larger than the fastening torque between the ultrasound transducer 52 and the fastener 13. As a result of this, in the case where the ultrasound transducer 52 is removed from the vibration transmission portion 12, fastening between the ultrasound transducer 52 and the fastener 13 is separated first before the fastening between the vibration transmission portion 12 and the fastener 13 is separated, so that the fastener 13 does not remain in the ultrasound transducer 52.

In particular, in a state in which the fastener 13 is fastened to the vibration transmission portion 12, it is possible to constitute a configuration such that the end surface of the second fastening portion 142 located on the distal end side Ar1 is brought into contact with the bottom surface of the second female screw portion 1232. In contrast, in a state in which the fastener 13 is fastened to the ultrasound transducer 52, it is possible to constitute a configuration such that the end surface of the first fastening portion 141 located on the proximal end side Ar2 is not brought into contact with the bottom surface of the first female screw portion 521. As a result of this, an axial force is generated in only the second fastening portion 142, so that it is possible to strengthen the fastening of the fastener 13 with respect to the vibration transmission portion 12. Therefore, in the case where the ultrasound transducer 52 is removed from the vibration transmission portion 12, it is possible to effectively avoid the fastener 13 from remaining in the ultrasound transducer 52.

Furthermore, the fastener described in Japanese Patent No. 6067208 does not include a tool gripping portion that functions as a screw head. Accordingly, the fastening portion needs to be gripped and fastened. In this case, there may be a case in which a portion that is gripped by the tool becomes deformed, and ultrasound vibration is not sufficiently transmitted, and the performance of the ultrasound treatment tool sometimes becomes unstable. In contrast, the fastener 13 according to the first embodiment includes the tool gripping portion 15 that functions as a screw head. Accordingly, it is possible to easily fasten the fastener 13 to the vibration transmission portion 12 by a desired torque without deforming the fastener 13, so that it is possible to stabilize the performance of the ultrasound treatment tool 2. Furthermore, the tool gripping portion 15 is removed after the fastening process due to a rupture of the rupture portion 17 as a result of a specific torque being applied to the rupture portion 17, so that the performance of the ultrasound treatment tool 2 is not affected.

Furthermore, with the fastener 13 according to the first embodiment, the cross-sectional areas of the cross sections of the main body 14, the tool gripping portion 15, and the connecting portion 16 obtained by cutting the main body 14, the tool gripping portion 15, and the connecting portion 16 by a plane orthogonal to the central axis Ax are smaller in the order of the tool gripping portion 15, the main body 14, and the connecting portion 16. As a result of this, a moment arm at the time of fastening of the fastener 13 with respect to the vibration transmission portion 12 can be made larger than a moment arm that is applied to the fastener 13 when the vibration transmission portion 12 is separated from the ultrasound transducer 52. Furthermore, the rupture portion 17 is constituted by a groove that is formed by cutting out a part of the connecting portion 16. As a result of this, it is possible to easily manage the torque for fastening the fastener 13 to the vibration transmission portion 12.

Here, in the case where a specific torque is denoted by T and a fastening torque between the ultrasound transducer 52 and the fastener 13 is denoted by T′, the ultrasound treatment tool 2 can be manufactured in a state in which Expression below (1) is satisfied.

$$\frac{T}{{T}^{′}} \geq 0.33 \qquad ( 1 )$$

Furthermore, effects obtained by manufacturing the ultrasound treatment tool 2 in the state in which Expression (1) is satisfied has been confirmed by the following experiment.

Specifically, in two cases out of a case in which T/T′ is set to 0.27, and a case in which T/T′ is set to 0.33, it is confirmed, after the ultrasound transducer 52 has been removed from the vibration transmission portion 12, whether the fastener 13 is separated from a position on the vibration transmission portion 12 side or the fastener 13 is separated from a position on the ultrasound transducer 52 side. Furthermore, in these two cases, the fastening torque T′ is set to be the same. The result of this is indicated by Table 1 below. Furthermore, in Table 1, a case in which the fastener 13 has been separated from the position on the vibration transmission portion 12 side is denoted by “OK”, whereas a case in which the fastener 13 has been separated from the position on the ultrasound transducer 52 side is denoted by “NG”.

TABLE 1

| T/T′ | Result |
|---|---|
| 0.27 | NG |
| 0.33 | OK |

### Modification 1-1 of the First Embodiment

In the first embodiment described above, the configuration of the fastener 13 may be changed. In a description below, for convenience of description, the fastener according to a modification 1-1 is referred to as a fastener 13A.

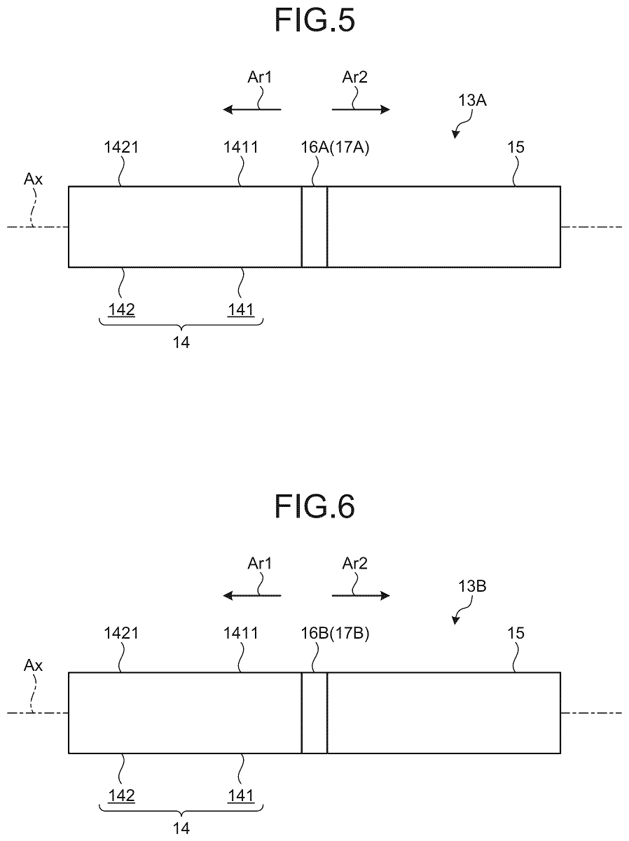

FIG. 5 is a diagram explaining the modification 1-1 of the first embodiment. Specifically, FIG. 5 is a schematic diagram of the fastener 13A according to the modification 1-1 when viewed from the side of the fastener 13A.

In the fastener 13A according to the modification 1-1, the configuration of each of the connecting portion 16 and the rupture portion 17 with respect to the fastener 13 that has been explained in the above described first embodiment is different. In a description below, for convenience of description, the connecting portion and the rupture portion according to the modification 1-1 are referred to as a connecting portion 16A (FIG. 5) and a rupture portion 17A (FIG. 5), respectively.

The connecting portion 16A according to the modification 1-1 is constituted by a different material from each of the main body 14 and the tool gripping portion 15. Specifically, the connecting portion 16A is constituted by an adhesive agent. In other words, the main body 14 and the tool gripping portion 15 are connected by the adhesive agent. Then, the connecting portion 16A functions as the rupture portion 17A, and is ruptured as a result of a specific torque being applied when the second fastening portion 142 is screwed together with the second female screw portion 1232.

Furthermore, in also the modification 1-1, it is possible to perform fine adjustment on the strength of the connecting portion 16A by adjusting the adhesion strength, and it is thus possible to easily manage the torque for fastening the fastener 13A to the vibration transmission portion 12.

Even when the above described fastener 13A according to the modification 1-1 is used, the same advantages as those provided in the above described first embodiment are provided.

### Modification 1-2 of the First Embodiment

The configuration of the fastener 13 used in the above described first embodiment may be changed. In a description below, for convenience of description, a fastener according to a modification 1-2 is referred to as a fastener 13B.

FIG. 6 is a diagram illustrating the modification 1-2 of the first embodiment. Specifically, FIG. 6 is a schematic diagram of the fastener 13B according to the modification 1-2 when viewed from the side of the fastener 13B.

With the fastener 13B according to the modification 1-2, a configuration of the connecting portion 16 and the rupture portion 17 with respect to the fastener 13 described above in the first embodiment is different. In a description below, for convenience of description, a connecting portion and a rupture portion according to the modification 1-2 are referred to as a connecting portion 16B (FIG. 6) and a rupture portion 17B (FIG. 6), respectively.

The connecting portion 16B according to the modification 1-2 is integrally constituted by the same material as that used for each of the main body 14 and the tool gripping portion 15, but, the strength of the connecting portion 16B is lower than the strength of each of the main body 14 and the tool gripping portion 15 as a result of heat treatment being performed by using a laser or the like. Moreover, the connecting portion 16B functions as the rupture portion 17B, and is ruptured as a result of a specific torque being applied when the second fastening portion 142 is screwed together with the second female screw portion 1232.

Furthermore, in also the modification 1-2, it is possible to perform fine adjustment on the strength of the connecting portion 16B by adjusting the heat treatment condition, and it is thus possible to easily manage the torque for fastening the fastener 13B to the vibration transmission portion 12.

Even when the above described fastener 13B according to the modification 1-2 is used, the same advantages as those provided in the above described first embodiment are provided.

### Modification 1-3 of the First Embodiment

The fastening structure between the fastener 13 and both the vibration transmission portion 12 and the ultrasound transducer 52 used in the above described first embodiment may be changed. In a description below, for convenience of description, a vibration transmission portion, an ultrasound transducer, and a fastener according to a modification 1-3 are referred to as a vibration transmission portion 12C, an ultrasound transducer 52C, and a fastener 13C, respectively.

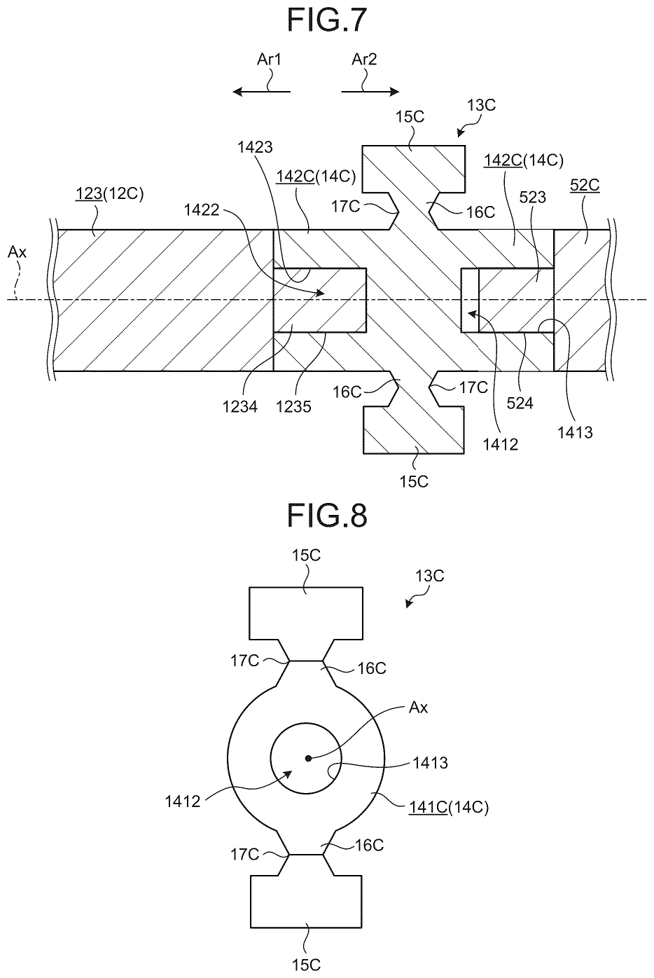

FIG. 7 and FIG. 8 are diagrams each explaining the modification 1-3 of the first embodiment. Specifically, FIG. 7 is a cross-sectional view of the fastening structure between the fastener 13C and both the vibration transmission portion 12C and the ultrasound transducer 52C when viewed from the side of the fastening structure. FIG. 8 is a diagram of the fastener 13C when viewed from the proximal end side Ar2 along the central axis Ax.

The structure of the end portion of the distal end side Ar1 of the ultrasound transducer 52C according to the modification 1-3 is different from that of the ultrasound transducer 52 described above in the first embodiment.

Specifically, the first female screw portion 521 described above in the first embodiment is not provided at the end portion of the ultrasound transducer 52C located on the distal end side Ar1. Furthermore, as illustrated in FIG. 7, on the end surface of the ultrasound transducer 52C located on the distal end side Ar1, a protrusion 523 that is formed in a columnar shape, that is coaxial with the central axis Ax, and that protrudes toward the distal end side Ar1 along the central axis Ax is provided. Furthermore, a screw groove 524 (FIG. 7) is provided on the outer circumferential surface of the protrusion 523. In other words, the protrusion 523 functions as a male screw portion. In a description below, for convenience of description, the protrusion 523 is referred to as a first male screw portion 523.

The structure of the end portion of the proximal end side Ar2 of the vibration transmission portion 12C according to the modification 1-3 is different from that of the vibration transmission portion 12 described above in the first embodiment.

Specifically, the second female screw portion 1232 described above in the first embodiment is not provided at the end portion of the vibration transmission portion 12C located on the proximal end side Ar2. Moreover, as illustrated in FIG. 7, on the end surface of the vibration transmission portion 12C located on the proximal end side Ar2, a protrusion 1234 that is formed in a columnar shape, that is coaxial with the central axis Ax, and that protrudes toward the proximal end side Ar2 along the central axis Ax is provided. Furthermore, a screw groove 1235 (FIG. 7) is provided on the outer circumferential surface of the protrusion 1234. In other words, the protrusion 1234 functions as a male screw portion. In a description below, for convenience of description, the protrusion 1234 is referred to as a second male screw portion 1234.

The fastener 13C according to the modification 1-3 includes, as illustrated in FIG. 7 or FIG. 8, a main body 14C, a tool gripping portion 15C, and a connecting portion 16C. The main body 14C, the tool gripping portion 15C, and the connecting portion 16C are integrally constituted by a material having predetermined rigidity, such as a titanium alloy.

The main body 14C has a columnar shape, is coaxial with the central axis Ax, and extends along the central axis Ax.

In the main body 14C, the portion of the proximal end side Ar2 functions as a first fastening portion 141C that is screwed together with the first male screw portion 523 included in the ultrasound transducer 52C.

Specifically, as illustrated in FIG. 7 or FIG. 8, on the end surface of the main body 14C located on the proximal end side Ar2, a recess portion 1412 that is recessed toward the distal end side Ar1 is provided. The recess portion 1412 has a circular shape in cross section that is obtained by cutting the recess portion 1412 by a plane orthogonal to the central axis Ax and that is coaxial with the central axis Ax, and has a substantially the same cross-sectional shape over the entire length along the central axis Ax. Furthermore, an inner diameter size of the recess portion 1412 is substantially the same as the outer diameter size of the first male screw portion 523. Furthermore, a screw groove 1413 is provided on the inner circumferential surface of the recess portion 1412 (FIG. 7 and FIG. 8). In other words, the first fastening portion 141C (the recess portion 1412) functions as a female screw portion.

Furthermore, in the main body 14C, a portion of the distal end side Ar1 functions as a second fastening portion 142C that is screwed together with the second male screw portion 1234 included in the vibration transmission portion 12C.

Specifically, as illustrated in FIG. 7, on the end surface of the main body 14C located on the distal end side Ar1, a recess portion 1422 that is recessed toward the proximal end side Ar2 is provided. The recess portion 1422 has a circular shape in cross section that is obtained by cutting the recess portion 1422 by a plane orthogonal to the central axis Ax and that is coaxial with the central axis Ax, and has a substantially the same cross-sectional shape over the entire length along the central axis Ax. Furthermore, an inner diameter size of the recess portion 1422 is substantially the same as the outer diameter size of the second male screw portion 1234. Furthermore, a screw groove 1423 is provided on the inner circumferential surface of the recess portion 1422 (FIG. 7). In other words, the second fastening portion 142C (the recess portion 1422) functions as a female screw portion.

The tool gripping portion 15C is arranged at a position opposite to the outer circumferential surface of the main body 14C and has a substantially rectangular parallelepiped shape. In the modification 1-3, two pieces of the tool gripping portions 15C are provided at 180-degree rotationally symmetric positions with respect to the central axis Ax. The two tool gripping portions 15C are the portions that are gripped by a tool from both of the upward and downward directions illustrated in FIG. 3 and FIG. 4 in the case where the second fastening portion 142 is screwed together with the second female screw portion 1232.

The connecting portion 16C is a portion that connects the main body 14C and each of the tool gripping portions 15C. In the modification 1-3, two pieces of the connecting portions 16C corresponding to the same number as the number of tool gripping portions 15C are provided. The connecting portions 16C are arranged between the outer circumferential surface of the main body 14C and the two respective tool gripping portions 15C, and have a columnar shape that linearly extends from the outer circumferential surface of the main body 14C toward the two respective tool gripping portions 15C from the outer circumferential surface of the main body 14C.

The cross-sectional areas of the cross sections of the main body 14C, the tool gripping portion 15C, and the connecting portion 16C described above obtained by cutting the main body 14C, the tool gripping portion 15C, and the connecting portion 16C by a plane including the central axis Ax are smaller in the order of the main body 14C, the tool gripping portion 15C, and the connecting portion 16C.

Then, in the modification 1-3, a rupture portion 17C is provided in each of the connecting portions 16C (FIG. 7 and FIG. 8).

Each of the rupture portions 17C is a portion that is ruptured as a result of a specific torque being applied to the associated rupture portion 17C when the second fastening portion 142C is screwed together with the second male screw portion 1234. In the modification 1-3, each of the rupture portions 17C is constituted by a groove that is formed by cutting out a part of the connecting portion 16C. More specifically, the rupture portions 17C that are the grooves are provided, on the outer circumferential surface of the respective connecting portions 16C, around the entire circumference of the respective connecting portions 16C in the circumferential direction with the central axis as a center.

Even when the fastening structure between the fastener 13C and both the vibration transmission portion 12C and the ultrasound transducer 52C according to the modification 1-3 described above are used, the same advantages as those provided in the above described first embodiment are provided.

### Modification 1-4 of the First Embodiment

The configuration of the fastener 13C used in the above described modification 1-3 may be changed. In a description below, for convenience of description, a fastener according to a modification 1-4 is referred to as a fastener 13D.

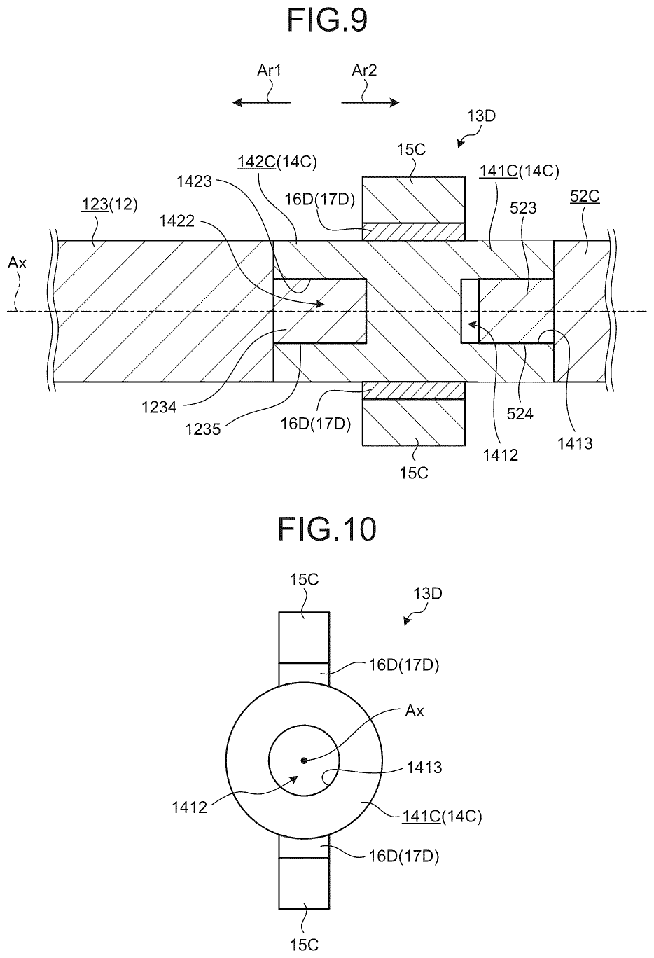

FIG. 9 and FIG. 10 are diagrams each explaining the modification 1-4 of the first embodiment. Specifically, FIG. 9 is a cross-sectional view that is associated with the cross-sectional view illustrated in FIG. 7. FIG. 10 is a diagram associated with that is associated with the diagram illustrated in FIG. 8.

In the fastener 13D according to the modification 1-4, the configuration of each of the connecting portion 16C and the rupture portion 17C with respect to the fastener 13C described above in the modification 1-3 is different. In a description below, for convenience of description, a connecting portion and a rupture portion according to the modification 1-4 are referred to as a connecting portion 16D (FIG. 9 and FIG. 10) and a rupture portion 17D (FIG. 9 and FIG. 10), respectively.

The connecting portion 16D according to the modification 1-4 has the same configuration as the configuration of the connecting portion 16A according to the above described modification 1-1 or has the same configuration as the configuration of the connecting portion 16B according to the above described modification 1-2. Moreover, the connecting portion 16D functions as the rupture portion 17D, and is ruptured as a result of a specific torque being applied when the second fastening portion 142C is screwed together with the second male screw portion 1234.

Even when the above described fastener 13D according to the modification 1-4 is used, the same advantages as those provided in the above described modification 1-3 are provided.

### Second Embodiment

In the following, a second embodiment will be described.

In a description below, the same components as those of the first embodiment described above are denoted by the same reference numerals, and detailed explanation thereof will be omitted or simplified.

In the second embodiment, the configuration of the fastener 13 is changed with respect to the above described first embodiment. In a description below, for convenience of description, an ultrasound treatment tool and a fastener according to the second embodiment are referred to as an ultrasound treatment tool 2E and a fastener 13E, respectively.

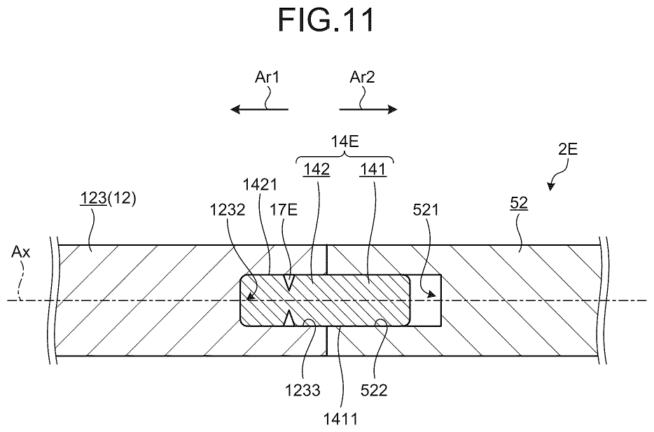

FIG. 11 is a diagram explaining the fastener 13E according to the second embodiment. Specifically, FIG. 11 is a cross-sectional view of the fastening structure between the fastener 13E and both the vibration transmission portion 12 and the ultrasound transducer 52 when viewed from the side of the fastening structure.

The fastener 13E according to the second embodiment includes, as illustrated in FIG. 11, a main body 14E and rupture portions 17E.

The main body 14E has the same configuration as that of the main body 14 described above in the first embodiment. In other words, the main body 14E includes, as illustrated in FIG. 11, the first fastening portion 141 (including the screw groove 1411) and the second fastening portion 142 (including the screw groove 1421) that are described above in the first embodiment.

Each of the rupture portions 17E are a portion that is ruptured as a result of a specific torque being applied to the associated rupture portion 17E when the first fastening portion 141 is screwed together with the second female screw portion 1232. In the second embodiment, as illustrated in FIG. 11, each of the rupture portions 17E is provided in the second fastening portion 142 in the main body 14E, and is constituted by a groove that is formed by cutting out a part of the second fastening portion 142. More specifically, each of the rupture portions 17E that corresponds to the groove is provided on the outer circumferential surface of the second fastening portion 142 around the entire circumference of the second fastening portion 142 in the circumferential direction with the central axis Ax as a center.

In the following, a method of manufacturing (assembly method) the ultrasound treatment tool 2E according to the second embodiment will be described with reference to FIG. 11.

Furthermore, in a description below, a step of connecting the vibration transmission portion 12 and the ultrasound transducer 52 by using the fastener 13E will mainly be described.

First, the worker screws the second fastening portion 142 together with the second female screw portion 1232. Then, the worker relatively rotates the vibration transmission portion 12 and the fastener 13E about the central axis Ax by using a tool for exclusive use, such as a torque wrench, and tighten the fastener 13E with respect to the vibration transmission portion 12. As a result of this, the fastener 13E is fastened to the second female screw portion 1232 at a specified torque. In this state, as illustrated in FIG. 11, the end surface of the second fastening portion 142 located on the distal end side Ar1 enters a state in which the end surface is in contact with the bottom surface of the second female screw portion 1232.

Then, the worker inserts the vibration transmission portion 12, to which the fastener 13E is fastened, into the interior of the housing 6, and then assembles the handpiece 4.

Then, the worker attaches the ultrasonic transducer unit 5 to the case body 61.

Then, the worker connects, by using the torque wrench, the vibration transmission portion 12 and the ultrasound transducer 52 (for example, see Japanese Patent No. 4675437).

Specifically, the worker allows the torque wrench to be engaged with the rotatable knob 9. Furthermore, the worker rotates the rotatable knob 9 about the central axis Ax by using the torque wrench. As a result of this, the vibration transmission portion 12 is rotated about the central axis Ax together with the rotatable knob 9. Then, the first fastening portion 141 in the fastener 13E that has been fastened to the vibration transmission portion 12 is screwed together with the first female screw portion 521, and is fastened to the first female screw portion 521 at a specified torque. In this state, the end surface of the vibration transmission portion 12 located on the proximal end side Ar2 and the end surface of the ultrasound transducer 52 located on the distal end side Ar1 abut against each other, and, as illustrated in FIG. 11, the end surface of the first fastening portion 141 located on the proximal end side Ar2 is not in contact with the bottom surface of the first female screw portion 521.

Here, in the case where the torque wrench is erroneously operated, or, in the case where a tool that is different from the torque wrench is used, it is assumed a case in which a specific torque that is equal to or larger than the above described specified torque is applied to each of the rupture portions 17E. In this case, each of the rupture portions 17E is ruptured. Moreover, even if the rotatable knob 9 is allowed to be rotated about the central axis Ax, the vibration transmission portion 12 enters an idle rotation state with respect to the ultrasound transducer 52. In other words, the ultrasound treatment tool 2E enters an unusable state.

According to the above described second embodiment, the following advantages are provided.

Incidentally, for example, in the case where the first fastening portion 141 is fastened to the ultrasound transducer 52 by a torque that exceeds the specific torque, the performance of treatment performed on the target region is changed from the desired performance.

In contrast, the fastener 13E according to the second embodiment includes the rupture portions 17E that are ruptured as a result of a specific torque being applied to the rupture portions 17E when the first fastening portion 141 is screwed together with the ultrasound transducer 52.

Consequently, in the case where the fastener 13E is fastened to the ultrasound transducer 52 by an erroneous operation performed by using a tool or the like that is different from the tool for exclusive use, such as a torque wrench, the fastener 13E is ruptured at the rupture portions 17E. Then, the ultrasound treatment tool 2E enters an unusable state.

Therefore, with the fastener 13E according to the second embodiment, it is possible to set the ultrasound treatment tool 2E to be an unusable state in the case where the fastener 13E is fastened to the ultrasound transducer 52 by an erroneous operation. In other words, it is possible to suppress an individual variation in the performance that occurs for each manufactured product of the ultrasound treatment tool 2E, and it is thus possible to stabilize the performance.

### Modification 2-1 of Second Embodiment

In the above described second embodiment, the configuration of the fastener 13E may be changed. In a description below, for convenience of description, a fastener according to a modification 2-1 is referred to as a fastener 13F.

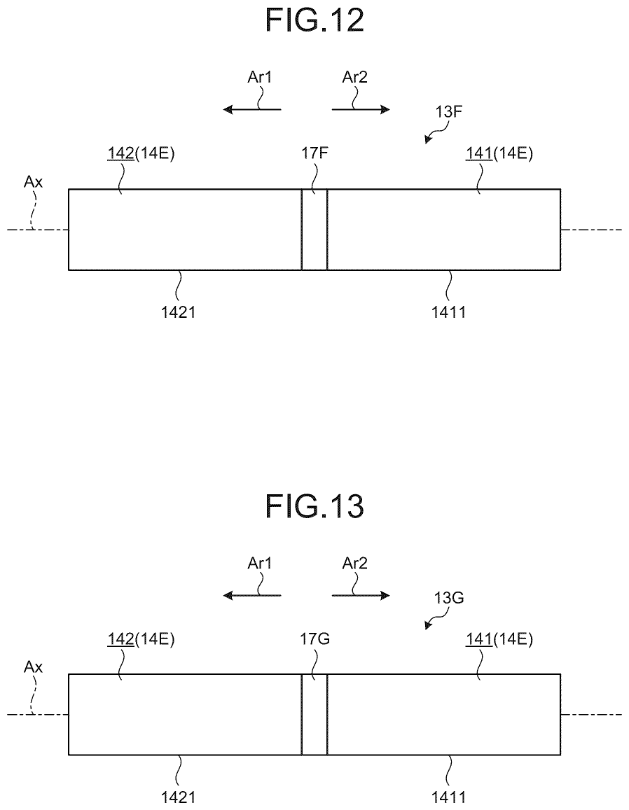

FIG. 12 is a diagram explaining the modification 2-1 of the second embodiment. Specifically, FIG. 12 is a schematic diagram of the fastener 13F according to the modification 2-1 when viewed from the side of the fastener 13F.

In the fastener 13F according to the modification 2-1, the configuration of each of the rupture portions 17E with respect to the fastener 13E described above in the second embodiment is different. In a description below, for convenience of description, a rupture portion according to the modification 2-1 is referred to as a rupture portion 17F (FIG. 12).

The rupture portion 17F according to the modification 2-1 is constituted by a different material from the main body 14E. Specifically, the rupture portion 17F is constituted by an adhesive agent that adheres two regions that are obtained by dividing the main body 14E by a plane orthogonal to the central axis Ax. Then, the rupture portion 17F is ruptured as a result of a specific torque being applied to the rupture portion 17F when the first fastening portion 141 is screwed together with the first female screw portion 521.

Even when the above described fastener 13F according to the modification 2-1 is used, the same advantages as those provided in the above described second embodiment are provided.

### Modification 2-2 of Second Embodiment

In the above described second embodiment, the configuration of the fastener 13E may be changed. In a description below, for convenience of description, a fastener according to a modification 2-2 is referred to as a fastener 13G.

FIG. 13 is a diagram explaining the modification 2-2 according to the second embodiment. Specifically, FIG. 13 is a schematic diagram of the fastener 13G according to the modification 2-2 when viewed from the side of the fastener 13G.

In the fastener 13G according to the modification 2-2, a configuration of each of the rupture portions 17E with respect to the fastener 13E described above in the second embodiment is different. In a description below, for convenience of description, a rupture portion according to the modification 2-2 is referred to as a rupture portion 17G (FIG. 13).

The rupture portion 17G according to the modification 2-2 is a region, in the main body 14E, that divides the main body 14E into two regions by a plane orthogonal to the central axis Ax. Moreover, in the region, strength is lower than that of the other region as a result of heat treatment being performed by using a laser or the like. Moreover, the rupture portion 17G is ruptured as a result of a specific torque being applied when the first fastening portion 141 is screwed together with the first female screw portion 521.

Even when the fastener 13G described above in the modification 2-2 is used, the same advantages as those provided in the above described second embodiment are provided.

### Modification 2-3 of the Second Embodiment

In the second embodiment described above, the fastening structure between the fastener 13E and both the vibration transmission portion 12 and the ultrasound transducer 52 may be changed. Furthermore, each of a vibration transmission portion and an ultrasound transducer according to a modification 2-3 has the same configuration as that of each of the vibration transmission portion 12C and the ultrasound transducer 52C described above in the modification 1-3. Furthermore, in a description below, for convenience of description, a fastener according to the modification 2-3 is referred to as a fastener 13H.

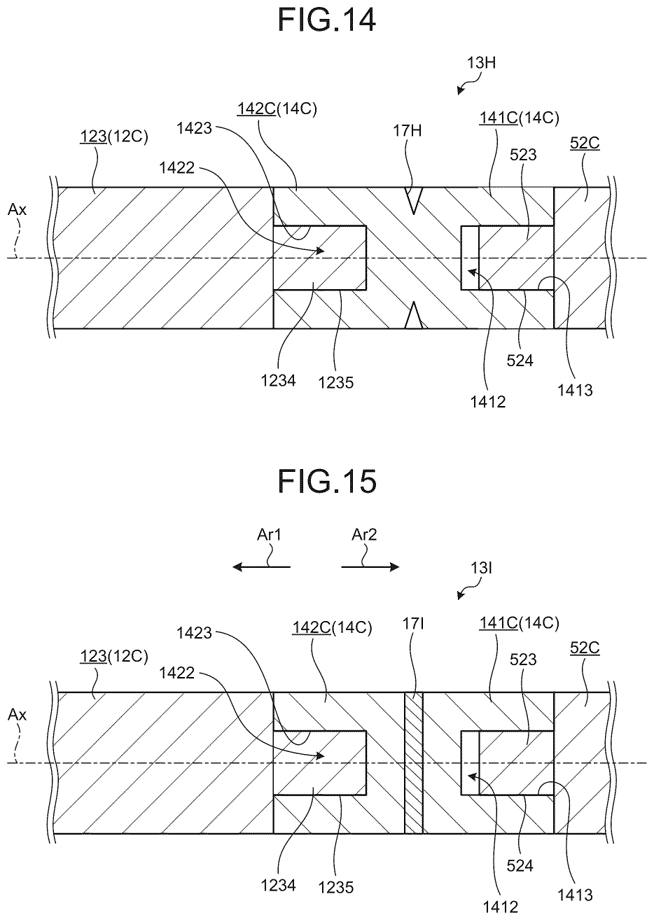

FIG. 14 is a diagram explaining the modification 2-3 according to the second embodiment. Specifically, FIG. 14 is a cross-sectional view of the fastening structure between the fastener 13H and both the vibration transmission portion 12C and the ultrasound transducer 52C when viewed from the side of the fastening structure.

The fastener 13H according to the modification 2-3 includes, as illustrated in FIG. 14, rupture portions 17H, in addition to the main body 14C described above in the modification 1-3.

Each of the rupture portions 17H is located at a boundary portion of the first and the second fastening portions 141C and 142C, and is ruptured as a result of a specific torque being applied to each of the rupture portions 17H when the first fastening portion 141C is screwed together with the first male screw portion 523. Each of the rupture portion 17H according to the modification 2-3 is constituted by a groove that is formed by cutting out a part of the main body 14C. More specifically, the rupture portions 17H that correspond to the grooves are provided, on the outer circumferential surface of the main body 14C, around the entire circumference of circumferential direction with the central axis Ax as a center.

Even when the fastening structure between the fastener 13H and both the vibration transmission portion 12C and the ultrasound transducer 52C described above in the modification 2-3 is used, the same advantages as those provided in the above described second embodiment are provided.

### Modification 2-4 of the Second Embodiment

In the above described modification 2-3, the configuration of the fastener 13H may be changed. In a description below, for convenience of description, a fastener according to a modification 2-4 is referred to as a fastener 13I.

FIG. 15 is a diagram explaining the modification 2-4 of the second embodiment. Specifically, FIG. 15 is a cross-sectional view associated with the cross-sectional view illustrated in FIG. 14.

In the fastener 13I according to the modification 2-4, the configuration of each of the rupture portions 17H with respect to the fastener 13H described above in the modification 2-3 is different. In a description below, for convenience of description, a rupture portion according to the modification 2-4 is referred to as a rupture portion 17I (FIG. 15).

The rupture portion 17I according to the modification 2-4 has the same configuration as that of the rupture portion 17F described above in the modification 2-1, or, has the same configuration as that of the rupture portion 17G described above in the modification 2-2. Moreover, the rupture portion 17I is ruptured as a result of a specific torque being applied when the first fastening portion 141C is screwed together with the first male screw portion 523.

Even when the fastener 13I according to the above described modification 2-4 is used, the same advantages as those provided in the above described modification 2-3 are provided.

### Other Embodiments

In the above, detailed description of the preferred embodiments have been described; however, the disclosure is not limited to only the embodiments described above.

In the above described embodiments, as the ultrasound treatment tool according to the disclosure, the configuration is constituted such that both of the ultrasound energy and the high frequency energy are applied to the target region; however, the configuration is not limited to this. As the ultrasound treatment tool according to the disclosure, a configuration in which only the ultrasound energy is applied to the target region may be used, or a configuration in which, in addition to the ultrasound energy, at least one of the high frequency energy and thermal energy is applied to the target region may be used. Here, the “thermal energy is applied to the target region” means that the heat generated in a heater or the like is transmitted to the target region.

In the first and the second embodiments described above, as the fastening structure between the fasteners 13 and 13E and both the vibration transmission portion 12 and the ultrasound transducer 52, only one of a fastening structure between the fasteners 13 and 13E and the vibration transmission portion 12 and a fastening structure between the fasteners 13 and 13E and the ultrasound transducer 52 may be used as the fastening structure that has been described above in the modification 1-3 or 2-3.

According to the fastener, a method of manufacturing an ultrasound treatment tool, and the ultrasound treatment tool according to the disclosure, it is possible to stabilize the performance of the ultrasound treatment tool.

Additional advantages and modifications will readily occur to those skilled in the art. Therefore, the disclosure in its broader aspects is not limited to the specific details and representative embodiments shown and described herein. Accordingly, various modifications may be made without departing from the spirit or scope of the general inventive concept as defined by the appended claims and their equivalents.

What is claimed is:

1. A fastener comprising:
a main body including
a first fastening portion configured to be screwed together with a ultrasound transducer configured to generate ultrasound vibration, and
a second fastening portion configured to be screwed together with a vibration transmission portion configured to transmit the ultrasound vibration, the main body being configure to connect the ultrasound transducer and the vibration transmission portion; and
a rupture portion configured to be ruptured as a result of a specific torque being applied when the first fastening portion is screwed together with the ultrasound transducer or when the second fastening portion is screwed together with the vibration transmission portion, wherein
in a state in which the first fastening portion is screwed together with the ultrasound transducer, an end surface of the first fastening portion is not in contact with a bottom surface of a fastening portion provided at the ultrasound transducer, and
in a state in which the second fastening portion is screwed together with the vibration transmission portion, an end surface of the second fastening portion is in contact with a bottom surface of a fastening portion provided at the vibration transmission portion.

2. The fastener according to claim 1, further comprising:
a tool gripping portion configured to be gripped by a tool; and
a connecting portion that connects the tool gripping portion and the main body, wherein
the rupture portion is provided at the connecting portion.

3. The fastener according to claim 2, wherein a cross-sectional area of the connecting portion is smaller than a cross-sectional area of each of the main body and the tool gripping portion.

4. The fastener according to claim 3, wherein the rupture portion is a groove that is formed by cutting out a part of the connecting portion.

5. The fastener according to claim 2, wherein the rupture portion is constituted by a different material from the main body and the tool gripping portion.

6. The fastener according to claim 2, wherein the rupture portion has a lower strength than each of the main body and the tool gripping portion as a result of heat treatment being performed.

7. The fastener according to claim 1, wherein the rupture portion is provided in the main body.

8. The fastener according to claim 7, wherein the rupture portion is provided at the second fastening portion.

9. The fastener according to claim 7, wherein the rupture portion is a groove that is formed by cutting out a part of the main body.

10. The fastener according to claim 7, wherein the rupture portion is constituted by a different material from the main body.

11. The fastener according to claim 7, wherein the rupture portion is a lower strength than the main body as a result of heat treatment being performed.

12. A method of manufacturing an ultrasound treatment tool that includes
an ultrasound transducer configured to generate ultrasound vibration,
a vibration transmission portion configured to transmit the ultrasound vibration, and
a fastener that connects the ultrasound transducer and the vibration transmission portion, the method comprising:
screwing the fastener together with the vibration transmission portion;
rupturing the fastener by applying a specific torque to the fastener when the fastener is screwed together with the vibration transmission portion; and
screwing the fastener together with the ultrasound transducer.

13. The method of manufacturing the ultrasound treatment tool according to claim 12, wherein
a female screw portion that is recessed toward a distal end of the vibration transmission portion is provided at a proximal end of the vibration transmission portion,
a male screw portion is provided at the fastener, wherein
at the screwing of the fastener together with the vibration transmission portion, the male screw portion is screwed together with the female screw portion while inserting the male screw portion into the female screw portion, and the fastener is in contact with a bottom surface of the female screw portion.

14. The method of manufacturing the ultrasound treatment tool according to claim 12, wherein
the fastener includes
a main body including
a first fastening portion configured to be screwed together with the ultrasound transducer, and
a second fastening portion configured to be screwed together with the vibration transmission portion,
a tool gripping portion configured to be gripped by a tool, and
a connecting portion configure to connect the tool gripping portion and the main body, wherein
at the screwing of the fastener together with the vibration transmission portion, the fastener is screwed together with the vibration transmission portion while gripping the tool gripping portion by the tool, and
at the rupturing of the fastener, the fastener is ruptured at the connecting portion.

15. The method of manufacturing the ultrasound treatment tool according to claim 12, wherein
a female screw portion that is recessed toward a proximal end of the ultrasound transducer is provided at a distal end of the ultrasound transducer, and
a male screw portion is provided at the fastener, wherein
at the screwing of the fastener together with the ultrasound transducer, the male screw portion is screwed together with the female screw portion while inserting into the female screw portion, and the male screw portion is set in a state in which the male screw portion is not in contact with a bottom surface of the female screw portion.

16. An ultrasound treatment tool comprising:
an ultrasound transducer configured to generate ultrasound vibration;
a vibration transmission portion configured to transmit the ultrasound vibration; and
a fastener configured to connect the ultrasound transducer and the vibration transmission portion, wherein
the fastener includes a main body that includes
a first fastening portion configured to be screwed together with the ultrasound transducer, and
a second fastening portion configured to be screwed together with the vibration transmission portion, wherein
an end surface of the first fastening portion is not in contact with a bottom surface of a fastening portion provided at the ultrasound transducer, and
an end surface of the second fastening portion is in contact with a bottom surface of a fastening portion provided at the vibration transmission portion.

17. The ultrasound treatment tool according to claim 16, wherein a rupture mark is provided on an outer surface of the main body.

18. The ultrasound treatment tool according to claim 16, wherein a rupture mark is provided at the first fastening portion.
